(19) United States
(12) Patent Application Publication
(10) Pub. No.: US 2023/0133761 A1
(43) Pub. Date: May 4, 2023

(54) INHIBITION OF FKBP1A FOR THE TREATMENT OF TRIPLE-NEGATIVE MAMMARY CARCINOMA

(71) Applicant: Heinrich-Heine-Universität Düsseldorf, Düsseldorf (DE)

(72) Inventors: Knud Esser, Köln (DE); Andrea Kulik, München (DE); Tanja Fehm, Düsseldorf (DE); Judith Steimel, Essen (DE)

(21) Appl. No.: 17/801,547

(22) PCT Filed: Mar. 11, 2021
(86) PCT No.: PCT/EP2021/056164

§ 371 Date: Aug. 23, 2022

(30) Foreign Application Priority Data
Mar. 12, 2020 (DE) 10 2020 203 224.6
Jun. 25, 2020 (DE) 10 2020 207 900.5

(51) Int. Cl. G01N 33/50; G01N 33/574

(52) U.S. Cl. CPC G01N 33/5011; G01N 33/57415; G01N 2500/10; G01N 2333/99

(57) ABSTRACT

The invention relates to a method for finding inhibitors of the peptidyl-prolyl cis-trans isomerase FKBP1A or antibodies, proteins or molecules having a specific affinity to FKBP1A. The invention also relates to FKBP1A-specific siRNA, inhibitors of the expression of FKBP1A, inhibitors of the enzymatic activity of FKBP1A, and inhibitors of the interaction(s) of FKBP1A with interaction partner(s), in each case for the treatment of diseases, in particular cancers or neurodegenerative diseases. The invention further relates to the use of FKBP1A as a prognostic or diagnostic marker for cancers. The cancers are preferably mammary carcinoma, in particular triple-negative mammary carcinoma, in this case very particularly the mesenchymal stem-like sub-type.

Priorities are claimed from German Patent Application No. 10 2020 203 224.6, filed Mar. 12, 2020, and German Patent Application No. 10 2020 207 900.5, filed Jun. 25, 2020.

The invention relates to a method for finding inhibitors of the peptidyl prolyl cis-trans isomerase FKBP1A or antibodies, proteins or molecules having a specific affinity to FKBP1A. The invention also relates to FKBP1A-specific siRNA, inhibitors of the expression of FKBP1A, inhibitors of the enzymatic activity of FKBP1A, and inhibitors of the interaction(s) of FKBP1A with interaction partner(s), in each case for the treatment of diseases, in particular cancers or neurodegenerative diseases. The invention further relates to the use of FKBP1A as a prognostic or diagnostic marker for cancers. The cancers are preferably mammary carcinoma, in particular triple-negative mammary carcinoma (TNBC, triple-negative breast cancer), very preferably the mesenchymal stem-like sub-type.

Triple-negative mammary carcinoma is diagnosed in 10-15% of all breast cancer patients and is characterized by a high recurrence rate, aggressive growth, early metastasis and a poor prognosis. The epigenetically misguided low differentiation status and the high stem cell character of the tumor cells are characteristic and probably one of the reasons for this high level of aggressiveness. This applies in particular to the mesenchymal stem-like sub-type of TNBC.

In contrast to other sub-forms of mammary carcinoma such as, for example, hormone receptor or Her2-positive mammary carcinoma, the current therapy for triple-negative mammary carcinoma according to the current guidelines consists exclusively of treatment with chemotherapeutic agents. However, these have a non-specific effect with significant side effects in healthy tissue. To date, no pharmacologically addressable cellular target structures are available for therapeutic approaches in triple-negative mammary carcinoma due to the negative expression of known oncological target structures (estrogen receptor, progesterone receptor or Her2 receptor) (cf. Kumar, P. & Aggarwal, R. An Overview of triple-negative breast cancer. Archives of Gynecology and Obstetrics (2016) doi:10.1007/s00404-015-3859-y).

Many targeted therapeutic approaches have been or are being investigated in clinical studies, such as, for example, the use of PARP inhibitors, angiogenesis inhibitors or mTOR inhibitors, or EGFR-directed treatment. However, these approaches showed only limited therapeutic efficacy (Fedele, P., Orlando, L. & Cinieri, S. Targeting triple-negative breast cancer with histone deacetylase inhibitors. Expert Opinion on Investigational Drugs (2017). doi:10.1080/13543784.2017.1386172). To date, no efficient personalized therapy for triple-negative mammary carcinoma has been developed.

Epigenetic agents, such as HDAC inhibitors, are also in the first clinical trials for the treatment of triple-negative mammary carcinoma. The approach of using epigenetic agents to address the high stem cell character, the low apoptosis induction and the low immunogenicity of the cancer cells is said to have great therapeutic potential (Fedele, P., Orlando, L. & Cinieri, S. Targeting triple-negative breast cancer with histone deacetylase inhibitors, Expert Opinion on Investigational Drugs (2017) doi:10.1080/13543784.2017.1386172 Mazzone R., Zwergel C., Mai A. & Valente S. Epi-drugs in combination with immunotherapy: a new avenue to improve anticancer efficacy Clinical Epigenetics (2017) doi: 10.1186/513148-017-0358-y). However, the agents currently being investigated only target general cellular epigenetic processes and are not very tumor-specific (Roberti, A., Valdes, A. F., Torrecillas, R., Fraga, M. F. & Fernandez, A. F. Epigenetics in cancer therapy and nanomedicine. Clinical Epigenetics (2019).doi:10.1186/s13148-019-0675-4).

US 2009/0215812 relates to compositions and methods for assessing the likelihood that a tumor will respond to an mTOR inhibitor, e.g. rapamycin or a rapamycin analog.

US 2016/0235731 relates to bifunctional compounds that act as protein degradation inducing units and methods for the targeted degradation of endogenous proteins by using said bifunctional compounds that bind a cereblon-binding moiety to a ligand capable of binding to the target protein, which can be used in the treatment of proliferative disorders.

US 2019/0092788 relates to 32-desoxo-rapamycin derivatives and their method of use.

WO 2009/030770 relates to methods and tools for obtaining an efficient prognosis (prognosis) of breast cancer estrogen receptor (ER)-negative patients, where the immune response is the key in mammary carcinoma prognosis.

WO 2013/093493 relates to a new rapamycin analogue, a method for its production and its use in therapy, in particular for the treatment of lupus and/or multiple sclerosis (MS).

S. Siamakpour-Reihani, PLoS ONE, 2011, Vol. 6, Art. e20412 relates to the immunosuppressive drug tacrolimus (FK506), which is able to inhibit SFRP2 and VEGF-stimulated in vitro tube formation and inhibit the migration of endothelial and breast cancer cells. Tacrolimus may be particularly useful in the treatment of breast cancer as it attenuates breast tumor xenograft growth in vivo and FKBP12 is expressed as an adapter molecule to calcineurin in breast carcinoma vasculature.

The aim of future personalized cancer therapy is a treatment with few side effects and aimed at the tumor, which specifically addresses pathophysiological changes in the cancer cells. There is a great need for novel, targeted therapy approaches in triple-negative mammary carcinoma, which do not attack the healthy tissue, but only specifically the tumor cells.

It is an object of the invention to provide new approaches to the therapy of cancers, in particular to the therapy of triple-negative mammary carcinoma, which have advantages over the prior art. The therapeutic approaches should be targeted, i. e. only attack the tumor cells and not the healthy tissue. In addition, it is an object of the invention to provide new diagnostic approaches for cancers, in particular for triple-negative mammary carcinoma, which have advantages over the prior art.

These objects are achieved by the subject matter of the claims.

Surprisingly, it was found that the peptidy prolyl cis-trans isomerase “FK506 binding protein 1a” (FKBP1A) is suitable as a tumor-specific target structure in triple-negative mammary carcinoma, FKBP1A opens up a wide range of possibilities for new, targeted therapy approaches and also for new diagnostic approaches.

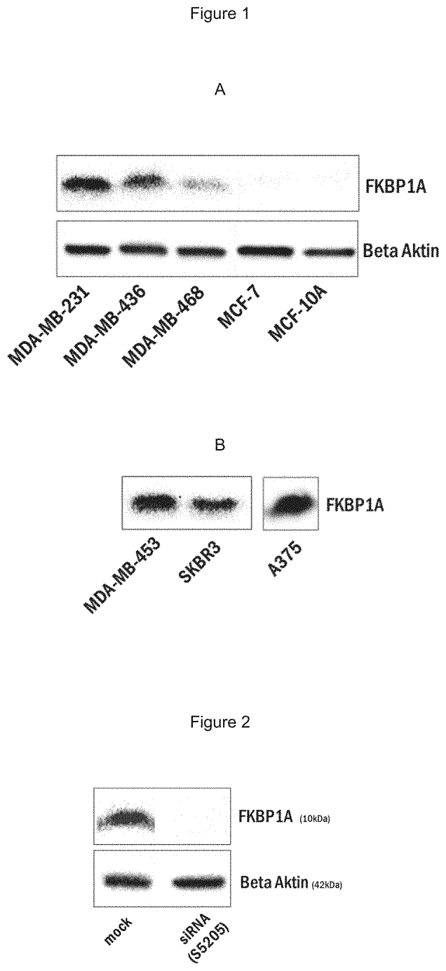

In various tumor cell models it could be shown that FKBP1A is expressed to a high degree in triple-negative mammary carcinoma cells, in particular in the mesenchymal stem-like sub-type, but is not detectable in hormone receptor-positive mammary carcinoma cells or in non-malignant cells (cf. FIG. 1A). In addition to tumor models of triple-negative mammary carcinoma, the high expression of FKBP1A could also be shown in cells of Her2-positive, hormone receptor-negative mammary carcinoma and malignant melanoma (see FIG. 1B).

It was surprisingly found that among the triple-negative breast cancer cells, those expressing FKBP1A to a particularly high degree are of the mesenchymal stem-like sub-type, i. e. have a high stem cell character, such as, for example, MDA-MB-231 and MDA-MB-436, which are commercially available. Thus, breast cancer cells of the mesenchymal stem-like sub-type are particularly well suited for the method according to the invention, since they enable good discrimination between effective and ineffective inhibitors.

To date, only the negative protein expression of FKBP1A in luminal MCF-7 cells or in healthy breast tissue is known (see https://www.proteinatlas.org/ENSG00000088832-FKBP1A/pathology, https://www.proteinatlas.org/ENSG00000088832-FKBP1A/tissue).

Furthermore, it was surprisingly found that FKBP1A plays an essential role in bringing about the inhibition of the physiological differentiation of triple-negative breast cancer cells, which is still poorly understood in tumor pathogenesis, and in arresting the cancer cells in a poorly differentiated state. Should it turn out in additional investigations that FKBP1A is also expressed in cells of the tumor microenvironment, such as, for example, in tumor-associated fibroblasts, macrophages or especially stem cells, especially mesenchymal stem cells, a novel therapeutic agent based on this therapeutic approach would thus address an increased expression not only in tumor cells, but also in cells of the tumor microenvironment and thereby further enhance the anti-tumor effect (He, W. et al. MSC-regulated lncRNA MACC1-AS1 promotes sternness and chemoresistance through fatty add oxidation in gastric cancer, Oncogene (2019), doi:10.1038/s41388-019-0747-0). In addition, due to the characteristic high stem cell character of the cancer cells of triple-negative breast cancer, here in particular the mesenchymal stem-like sub-type, the inventors expect a role of FKBP1A in so-called tumor stem cells of other cancers that is comparable to that described here. In particular, cancer cells of the mesenchymal stem-like sub-type, such as, for example, MDA-MB-231 have a particularly high proportion of tumor stem cells. Tumor stem cells are a subpopulation of cancer cells, which in tumor pathophysiology of cancer are considered to play an important role in terms of therapy resistance and metastasis (Kuşo\check{g}lu, A. & Biray Avci, Ç. Cancer stem cells: A brief review of the current status, Gene (2019), doi:10.1016/j.gene.2018.09.052). A FKBP1A-targeted therapy is novel, therefore, not only specific against cancer cells of triple-negative mammary carcinoma and here especially against tumor stem cells, but also against the subpopulation of tumor stem cells of other cancers.

In addition, it was surprisingly found that FKBP1A can be characterized and addressed therapeutically as a tumor-specific target in triple-negative breast cancer, in particular of the mesenchymal stem-like sub-type, via a directed expression reduction of FKBP1A using specific siRNA (see FIG. 2), This is also important for current immunotherapy (Bu, X., Yao, Y. & Li, X. Immune checkpoint blockade in breast cancer therapy: in Advances in Experimental Medicine and Biology (2017). doi:10.1007/978-981-10-6020-5_18). Thus, by using the therapy approach according to the invention as an adjuvant, current therapy can be substantially improved, in particular in synergistic treatment together with immunotherapy by

targeted reduction of the cellular (tumor) stem cell character of the cancer cells,

reduction of mesenchymal character of cancer cells,

increase in the cellular differentiation status,

enhancement of cellular apoptosis induction, and

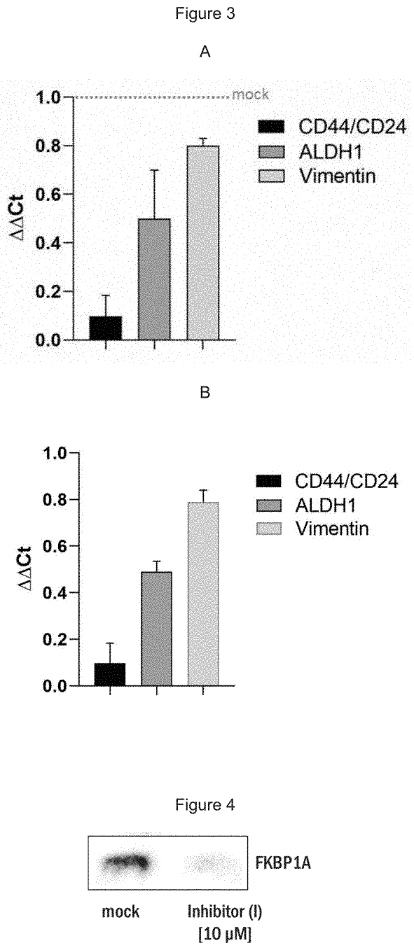

increase in cellular immunogenicity, (see FIG. 3).

According to the invention, personalized medicine in the treatment of triple-negative mammary carcinoma is made possible by using siRNAs, antibodies or pharmaceuticals which are each specific or selective for FKBP1A.

The tumor cells of TNBC, especially of the mesenchymal stem-like sub-type, have a particularly high tendency to metastasize. In addition to the high tumor stem cell character described, these tumor cells have a particularly high mesenchymal cell characteristic and are characterized by the high expression of markers of the epithelial-mesenchymal transition (EMT). Surprisingly, it was found that downregulation of FKBP1A leads to a reduction in EMT markers such as vimentin (see FIG. 3A). In addition, it was surprisingly found that in mammary carcinoma, the expression of FKBP1A correlates with the expression of TGF-beta receptor II, an essential inducer of EMT by binding to its ligand TGF-beta. For this reason, a high expression of FKBP1A is expected in single tumor cells or in groups of tumor cells invading the extracellular matrix surrounding the tumor. A FKBP1A-targeted therapy is novel, therefore, not only specific against cancer cells of triple-negative mammary carcinoma, but also specific against the subpopulation of (in the case of epithelial cells: after an EMT) cancer cells invading the tumor extracellular matrix (also of originally FKBP1A-negative or low-expressing tumor tissue).

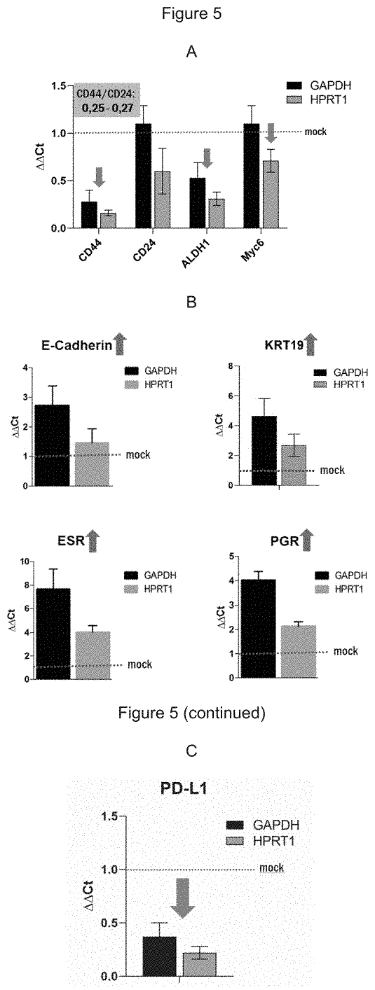

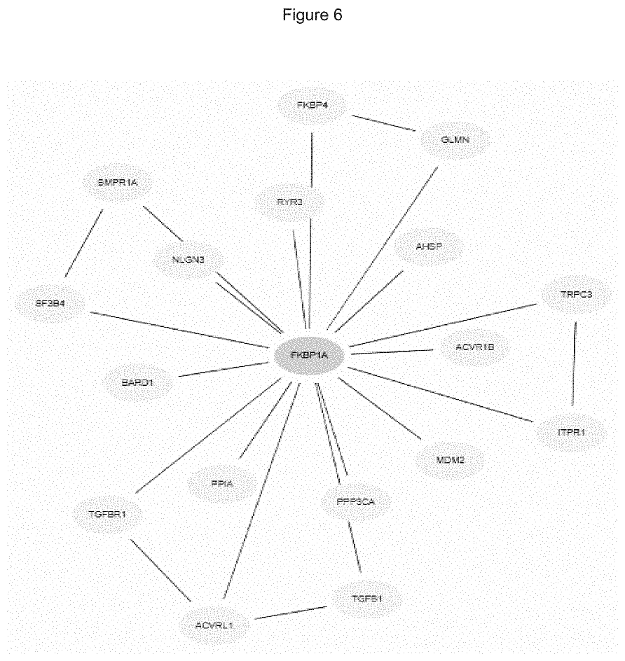

For initial drug candidates, it was experimentally shown that they induce a reduction in expression of FKBP1A in triple-negative mammary carcinoma (see FIG. 4) and also bring about a reduction in the expression of (tumor) stem cell markers (see FIG. 5). In addition, they have been found to induce metabolic reprogramming. For certain macrolides (e. g, rapamycin or rapamycin derivatives such as, for example, everolimus (RAD001), temsirolimus (001-779) or ridaforolimus (AP23573)), some of which are approved for antibiotic therapy in humans, or for FK506 there is an inhibitory effect described against FKBP1A. These inhibitors address mTOR or calcineurin via FKBP1A as an adapter molecule. In addition, specific inhibitors of FKBP1A have been developed in the context of neurological therapy approaches, which; in contrast to rapamycin or FK506, do not additionally bind to mTOR or calcineurin, but address FKBP1A only and therefore have no immunosuppressive effect. This is of great importance, particularly in the context of the synergistic immunotherapies of triple-negative mammary carcinoma that are being sought here. In addition, the anti-tumor effect described relates to an isolated addressing of FKBP1A, since an interaction of FKBP1A with these proteins has not been described without the mTOR-specific or calcineurin-specific inhibitors mentioned. These inhibitors which address FKBP1A activity only include, inter alia, ElteN378, V-10367 (Vertex, USA), GPI-1046 (Guilford, USA), GPI-1485 (Guilford, USA), SLF, FK1706, FK-1012, AG5437 or AG5507 (Kolas, J. M., Voll, A. M., Bauder, M. & Hausch, F. FKBP Ligands—Where We Are and Where to Go? Front. Pharmacol. (2018), doi:10.3389(fphar.2018.01425). The use of such and other inhibitors for cancer therapy, especially for the therapy of triple-negative mammary carcinoma in an epigenetic context, could represent a significant improvement in targeted cancer therapy.

Contrary to US 200910215812, it was surprisingly found that mTOR inhibitors have no significant effect in the treatment of TNBC. Also in cell culture, mTOR inhibitors show no effect in models of TNBC, in particular of the mesenchymal stem-like sub-type; such as in MDA-MB-231 cells. Experimental findings even show a reduction in apoptosis induction after incubation of MDA-MB-231 cells with rapamycin derivatives. Since FKBP1A is strongly expressed, particularly in the mesenchymal-stem-like sub-type of TNBC, such as, for example, MDA-MB-231 cells, the correlation of FKBP1A expression and anti-tumor effect postulated in US 2009/0215812 by rapamycin derivatives especially for the mesenchymal sub-type cannot be confirmed. US 2009/0215812 describes increased anti-tumor activity by FKBP1A when FKBP1A is expressed to an increased extent in the tumor cells, i. e. precisely the opposite of the correlation according to the invention. In addition, US 2009/0215812 does not show any pro-cancerogenic effect in correlation with the sole expression or activity of FKBP1A, in particular with regard to the tumor stem cell characteristics and/or inducers of EMT, in particular not in TNBC and especially not in the mesenchymal stem-like sub-type.

In addition, contrary to Siamakpour-Reihani et al., it was surprisingly found that there is a pro-cancerogenic effect in correlation with the sole expression or activity of FKBP1A, in particular with regard to the tumor stem cell characteristics and/or inducers of EMT, in particular in TNBC and especially in the mesenchymal stem-like sub-type. The anti-tumor effect of the FKBP1A-binding molecules (inter alia, tacrolimus) described by Siamakpour-Reihani et al. is described exclusively via the binding to the target molecule calcineurin, in which FKBP1A only serves as an adapter molecule for tacrolimus.

US 2019/0092788 describes the expression of FKBP1A in tumor cells as an indicator of successful inhibition of the mTOR signaling pathway via rapamycin derivatives. Derivatives are described whose mTOR inactivation is related to an expression of FKBP1A to varying degrees. An anti-tumor effect through direct and sole addressing of FKBP1A is not described, especially not with regard to the tumor stem cell characteristics and/or inducers of EMT, especially not with TNBC and especially not with the mesenchymal stem-like sub-type.

WO 2013/093493 describes rapamycin derivatives which, in addition to the binding and inhibition of mTOR, show different degrees of inhibition of the PPI of FKBP1A. It is demonstrated that derivatives that cause stronger PPI inhibition of FKBP1A show increased inhibition of cell growth of SF268, U87MG (both glioblastoma), DU145 and PC3 (both prostate carcinoma). Very high concentrations of the inhibitors in the micromolar range are used in this case, while a strong effect has already been shown in the sub-nanomolar range for rapamycin derivatives. An off-target effect seems likely in this context. An anti-tumor effect by direct and sole addressing of FKBP1A with regard to tumor stem cell characteristics and/or inducers of EMT is not described, especially not in the case of TNBC and especially not in the case of the mesenchymal stem-like sub-type. No significant growth inhibition could be shown by isolated addressing of, in particular, the enzymatic activity FKBP1A in the tumor cells. In addition, experimental findings indicate that the anti-tumor effect described could be independent of the PPI activity of FKBP1A, in contrast to WO 2013/093493.

US 2016/0235731 shows data on reducing the cellular protein concentration of FKBP1A knockdown using FKBP1A-addressing PROTCAs in the AML cell line MV4-11. An anti-tumor effect of the knockdown is not shown. Reference is only made to the well-known oncogenic signaling of FKBP1A. It can be assumed that FKBP1A inhibits TGF-beta receptor I activity. In contrast, it was surprisingly found that FKBP1A is particularly highly expressed in TNBC and especially in the mesenchymal stem-like sub-type, for which it is known that TGF-beta induces a strong epithelial-mesenchymal transition via activation of the TGF-beta receptors, According to the scientifically known data, a targeted reduction in FKBP1A expression or concentration, for example by an FKABP1A-addressing PROTAC as described in US 2016/0235731, should lead to a reduction in TGF-beta receptor inactivation by FKBP1A especially in TNBC and especially in the mesenchymal stem-like sub-type and thus to increased TGF-beta signaling with a known pro-oncogenic effect in TNBC.

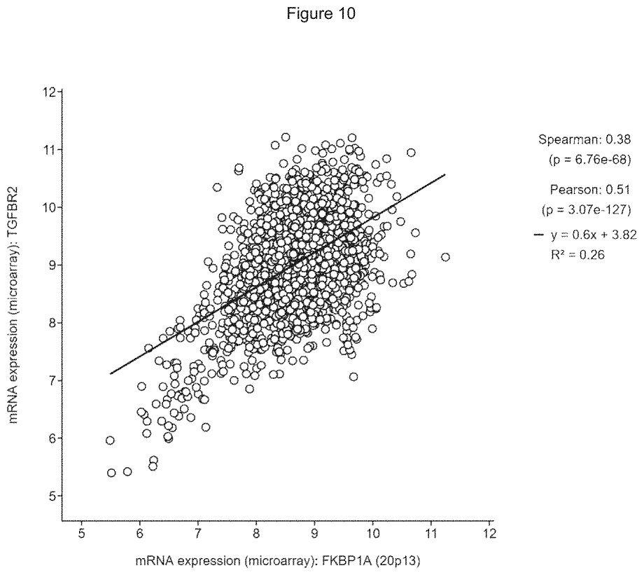

Surprisingly, the degradation of FKBP1A induces a reduction in mesenchymal cell character and MET. This has not yet been described in the literature, particularly for triple-negative breast cancer. Surprisingly, a strong correlation of the expression of FKBP1A with the TGF-beta receptor II could be demonstrated in breast cancer cells for the first time (see FIG. 10). However, this correlation could not be demonstrated for TGF-beta receptor I. TGF-beta-activated TGF-beta receptor II transphosphorylates TGF-beta receptor I and induces EMT, particularly in TNBC and especially “the mesenchymal stem-like” sub-type. These data suggest for the first time that FKBP1A mediates induction of EMT via high expression of TGF-beta receptor II and possibly also interactions via activation of TGF-beta receptor I. These findings have not been described to date and thus represent a novel connection between the high expression of FKBP1A, especially in TNBC and in particular the “mesenchymal stem-like” sub-type.

The correlation of a disease prognosis with the expression of FKBP1A within the defined immune module, which is described in WO 2009/030770 for ER- and HER2-negative breast tumors, is presented as a positive correlation. Accordingly, increased expression of FKBP1A is associated with a better prognosis for the patient and a lower probability of metastasis, and in isolation it is related to better immunogenicity of the cancer cells and the resulting spread of the tumor, which is better prevented by the immune system, and therefore less metastasis. Comparably, a correlation of the expression of FKBP1A with a better prognosis for the patients and the associated lower probability of metastasis is also described for HER2+ tumors. Since both tumor types, in particular TNBC, are characterized by a high level of malignancy, it is not clear from WO 2009/030770 why FKBP1A, associated here with a better prognosis, can be used for precise typing of these breast cancer sub-types. In contrast, it was surprisingly found that increased expression of FKBP1A, particularly in TNBC, especially in the mesenchymal stem-like stub type, is associated with increased expression of stem cell and EMT markers, from which, in contrast to WO 20091030770, increased invasion of cancer cells, metastasis and thus worse prognosis results. The present invention shows for the first time a use as a prognostically unfavorable marker, particularly in TNBC.

Another form of future targeted therapy is the development of so-called PROTAC (PROteolysis-TArgeting Chimeras) (An, S. & Fu, L. Small-molecule PROTACs: An emerging and promising approach for the development of targeted therapy drugs, EBioMedicine (2018), doi:10.1016/j.ebiom.2018.09.005). Here, the target molecule is not inhibited, but degraded via specific molecules by means of targeted cellular degradation. FKBPs, in particular FKBP1A, are frequently used fusion partners in this case. This approach can also be used directly to inactivate FKBPs, preferably FKBP1A, by means of degradation in the treatment of triple-negative mammary carcinoma. An example of such an agent is FKBP12 PROTAC RC32 (CAS No.: 2375555-66-9). SLF (CAS No.: 195513-96-3) can be used as a precursor for the synthesis of a FKBP-specific PROTACs. One advantage of the targeted protein degradation induced by PROTAC compared to inhibition of the enzyme function is that the proteolytic digestion of an enzymatic target structure such as FKBP1A in the cell mediated by PROTACs not only suppresses its enzymatic activity, but also any protein interactions (cf. [0030] and FIG. 6). For the first time, a stronger anti-tumor effect, inter alia, on the tumor stem cell and EMT markers mentioned, was determined by FKBP12 PROTAC RC32-mediated expression reduction compared to pure inhibition of the enzyme function of FKBP1A. A stronger anti-tumor effect (inter alia on cell proliferation) through expression reduction compared to pure inhibition of the enzyme function of FKBP1A in TNBC was also found for the first time in siRNA knock-down experiments against enzyme inhibitors. In addition, potential binding partners may also be degraded by PROTACs. The spectrum of effects is therefore much larger overall and therapy is more efficient. In addition, the immunogenicity of the tumor is increased by the presentation of proteolytically degraded antigens, which plays an important role in synergistic immunotherapy. The PROTAC technology can also be used for the therapy of triple-negative mammary carcinoma via synthesis of not only FKBP1A as an agent, but a dual-targeting agent for the simultaneous degradation of two target structures such as FKBP1A and PIN1 or retinoid receptors RAR or RXR (cf. [0062]). The design and the synthesis of such an agent are known to the skilled person.

In addition to FKBP1A, other representatives of the FK506-binding proteins are also potential target structures in triple-negative mammary carcinoma and tumor stem cells with the role described here for FKBP1A in the pathophysiology of cancer cells and with therapeutic approaches using specific inhibitors or other targeted addressing described here. Mention should be made here in particular of FKBP12.6, FKBP13, FKBP25, FKBP51 and FKPB52 (Kolos, J, M., Voll, A. M Bauder, M. & Hausch, F. FKBP Ligands—Where We Are and Where to Go? Front. Pharmacol. (2018)).

In addition and independently of a therapeutic application, diagnostic applications are also directed towards FKBP1A according to the invention. Since FKBP1A, in contrast to healthy breast tissue, is strongly expressed in triple-negative breast cancer cells, there is a high potential to use FKBP1A in the tissue and serum or plasma of patients a suitable test procedure for both primary diagnostics and therapeutic follow-up. In addition, FKBP1A is more highly expressed in “mesenchymal stem-like” triple-negative breast cancer cells, for which a particularly high stem cell character has been described, than in basal-like triple-negative breast cancer cells (see FIG. 2). Therefore, FKBP1A could be a diagnostic and prognostic marker for triple-negative breast cancer cells with a particularly strong epigenetically deregulated, low cell differentiation, low epithelial character and high aggressiveness of triple-negative tumors. Due to the high correlation to EMT markers, FKBP1A can also be used as a diagnostic and prognostic marker in tumor cells of the tumor extracellular matrix for invasive tumors.

FIG. 1 shows a western blot analysis of the protein expression of FKBP1A in different cells.

FIG. 2 shows the downregulation of FKBP1A protein expression by specific siRNA as a result of western blot analysis.

FIG. 3 shows the result of an expression analysis of (tumor) stem cell and mesenchymal markers after FKBP1A knock-down.

FIG. 4 shows downregulation of FKBP1A protein expression by inhibitor (I) as a result of western blot analysis of protein expression.

FIG. 5 shows the result of an expression analysis of (tumor) stem cell and epithelial differentiation markers and PD-L1 after expression reduction of FKBP1A using inhibitor (I).

FIG. 6 shows known interaction partners of FKBP1A (source: canSAR Black (https://cansarblack.icr.ac.uk/?type=protein,compound,cellline,disease).

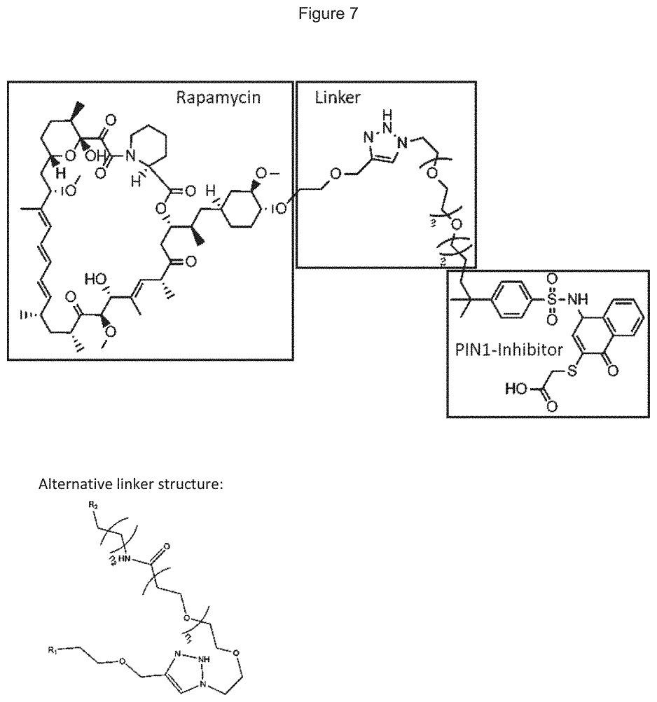

FIG. 7 shows an example of the theoretical structure of a dual-targeting agent against FKBP1A and PIN1.

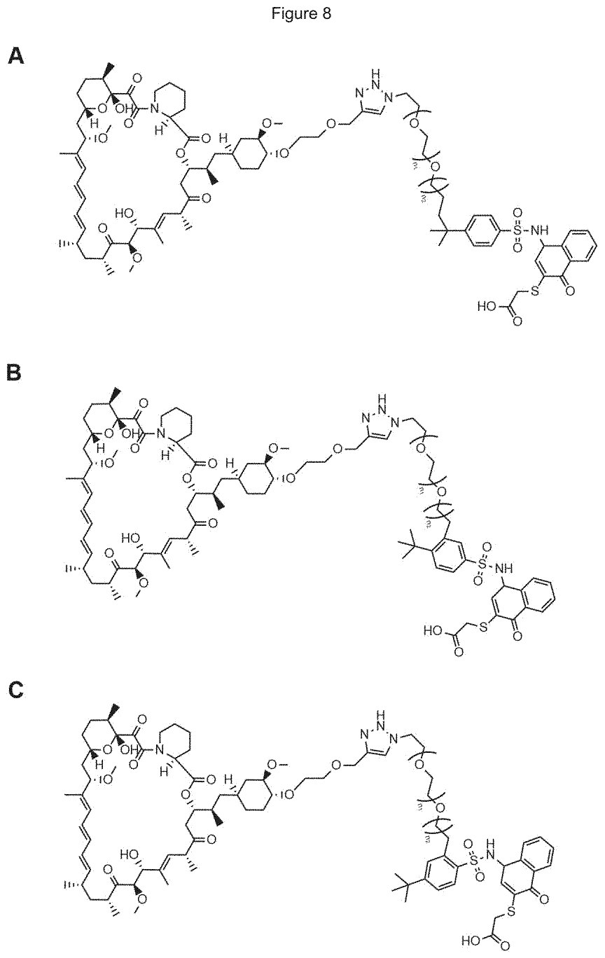

FIG. 8 shows examples, based on FIG. 7, of dual-targeting agents composed of rapamycin and KPT-6556.

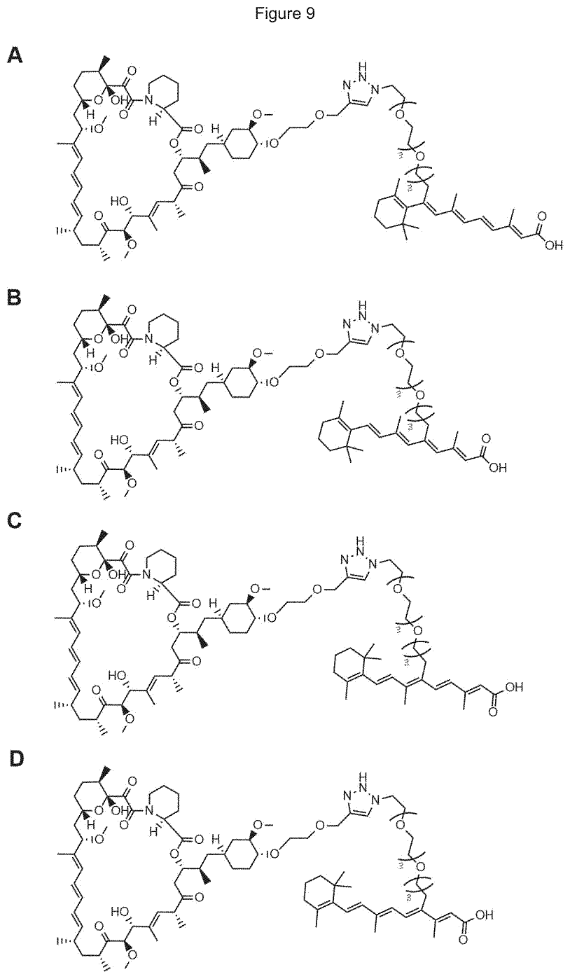

FIG. 9 shows examples, based on FIG. 7, of dual-targeting agents composed of rapamycin and ATRA.

FIG. 10 shows a co-expression analysis of FKBP1A and TGF-beta receptor II in primary breast cancer tissue.

One aspect of the invention relates to a method for finding inhibitors of the peptidyl prolyl cis-trans isomerase FKBP1A, which are suitable for the treatment of diseases; including neurodegenerative diseases such as, for example, Alzheimer's disease; in particular cancers; preferably triple-negative mammary carcinoma; Her2-positive hormone receptor-negative mammary carcinoma or malignant melanoma; particularly preferably triple-negative mammary carcinoma, comprising the steps of
(a) providing an inhibitor of
(i) the expression of FKBP1A; and/or
(ii) the enzymatic activity of FKBP1A; and/or
(iii) the interaction(s) of FKBP1A with interaction partner(s);
(b) incubating cancer cells of a cancer cell line with the inhibitor of FKBP1A, and determining a characteristic of the cancer cells;
(c) incubating cancer cells of the same cancer cell line as in step (b) in the absence of the inhibitor of FKBP1A and determining the same characteristic of the cancer cells as in step (b) under the same conditions as step (b);
(d) comparing the determined characteristic of the cancer cells according to steps (b) and (c).

Step (a) of the method according to the invention preferably comprises the sub-steps of
(a₁) providing several test substances;
(a₂) screening the test substances with regard to their inhibitory effect on
   (i) the expression of FKBP1A; and/or
   (ii) the enzymatic activity of FKBP1A; and/or
   (iii) interaction(s) of FKBP1A with interaction partner(s);
(a₃) selecting at least one screened test substance, the inhibitory effect of which is stronger than the inhibitory effect of at least one other screened test substance, and providing this selected test substance as an inhibitor of FKBP1A.

Typically, the cancer cell line is characteristic of triple-negative mammary carcinoma or is a cancer cell line of triple-negative mammary carcinoma, preferably selected from the group consisting of MDA-MB-231, MDA-MB-436 and MDA-MB-468, especially MD-MB-231. Suitable cancer cell lines are available, for example, from the American Type Culture Collection.

The characteristic of the cancer cells determined in step (b) is preferably selected from the group consisting of (A) cellular differentiation status, (B) epithelial characteristics, (C) cellular immunogenicity, (D) apoptosis induction or apoptosis ability, and (E) cellular stem cell character.

In preferred embodiments of the method according to the invention, the inhibitor provided in step (a) inhibits the interaction(s) of FKBP1A with interaction partner(s), with at least one interaction partner being selected from the group consisting of interaction partners which effect a reduction or arrest of (A) the cellular differentiation status, (B) the epithelial characteristics, (C) the cellular immunogenicity, (D) the induction of apoptosis or apoptosis ability, and/or (E) the cellular stem cell character.

In preferred embodiments of the method according to the invention, the inhibitor provided in step (a) inhibits the interaction(s) of FKBP1A with interaction partner(s), at least one interaction partner being selected from the group consisting of interacting proteins, interacting peptides, and interacting nucleic acids.

Numerous interaction partners of FKBP1A and their interactions are known to a person skilled in the art. According to the invention, preferred interaction partners of FKBP1A are selected from the group consisting of AHSP, cyclophilin-type peptidyl prolyl isomerases, FKBP-type peptidyl prolyl isomerases, inositol triphosphate receptors, MDM2/MDM4, phosphoprotein phosphatases, ryanodine receptors, SF3B4, TGF-beta, TKL Ser/Thr protein kinases and type B carboxylesterases/lipases. Interaction partners of FKBP1A that are preferred according to the invention are summarized in the following table (cf. FIG. 6):

| Surname | Uniprot ID | Family |
|---|---|---|
| ACVR1B | P36896 | TKL Ser/Thr protein kinases (tyrosine kinase |
| | | like) |
| ACVRL1 | P37023 | TKL Ser/Thr protein kinases (tyrosine kinase |
| | | like) |
| AHSP | Q9NZD4 | AHSP (alpha hemoglobin stabilizing protein) |
| BARD1 | Q99728 | |
| BMPR1A | P36894 | TKL Ser/Thr protein kinases (tyrosine kinase |
| | | like) |
| FKBP1A | P62942 | FKBP-type peptidyl prolyl isomerases (FK506 |
| | | binding protein) |
| FKBP4 | Q02790 | |
| GLMN | Q92990 | |
| ITPR1 | Q14643 | Inositol triphosphate (InsP3) receptors |
| MDM2 | Q00987 | MDM2/MDM4 (murine double minute 2/ |
| | | human homologue) |
| NLGN3 | Q9NZ94 | Type B carboxylesterases/lipases |
| PPIA | P62937 | Cyclophilin-type peptidyl prolyl isomerases |
| PPP3CA | Q08209 | Phosphoprotein phosphatases |
| RYR3 | Q15413 | Ryanodine receptors (TC 1.A.3.1) |
| SF3B4 | Q15427 | SF3B4 (splicing factor 3B subunit 4) |
| TGFB1 | P01137 | TGF-beta (transforming growth factor beta) |
| TGFBR1 | P36897 | TKL Ser/Thr protein kinases (tyrosine kinase |
| | | like) |
| TRPC3 | Q13507 | TRP receptors (TC 1.A.4) (tryptophan transient |
| | | receptor) |

In preferred embodiments of the method according to the invention, the characteristic of the cancer cells is determined in step (b) by quantifying differentiation markers (cell type markers).

Suitable differentiation markers and methods for their respective quantification are known to a person skilled in the art. In this context, for example, full reference can be made to: Akrap, N. et al, Identification of Distinct Breast Cancer Stem Cell Populations Based on Single-Cell Analyzes of Functionally Enriched Stem and Progenitor Pools. Stem Cell Reports (2016). doi:10.1016/j.stemcr.2015.12.006; Prabhakaran P., Hassiotou F., Blancafort P. & Filgueira L. Cisplatin induces differentiation of breast cancer cells. Front. Oncol. (2013). doi:10.3389/fonc.2013.00134.

Suitable differentiation markers are, for example, CD3 on T lymphocytes, CD14 on monocytes, CD16 and CD56 on NK cells and granulocytes, CD19 and CD20 on B lymphocytes. Differentiation markers for mesenchymal stem cells are, for example, CD13, CD29, CD44, CD49e, CD54, CD71, CD73, CD90, CD105, CD106, CD166 and HLA-ABC. The differentiation markers are preferably those of epithelial cells.

In preferred embodiments of the method according to the invention, the characteristic of the cancer cells is determined in step (b) by quantifying epithelial markers.

Suitable epithelial markers and methods for their respective quantification are known to a person skilled in the art. In this context, for example, full reference can be made to Frixen, U. H. et al. E-cadherin-mediated cell-cell adhesion prevents invasiveness of human carcinoma cells. J Cell Biol (1991). doi:10.1083/jcb.113.1.173.

Epithelial markers (epithelial cell markers) preferred according to the invention are selected from the group consisting of cytokeratins, cell adhesion molecules and cell surface proteins. A preferred cell adhesion molecule according to the invention, which can be used as an epithelial marker, is E-cadherin.

In preferred embodiments of the method according to the invention, the characteristic of the cancer cells is determined in step (b) by quantifying mesenchymal markers.

Suitable mesenchymal markers and methods for their respective quantification are known to a person skilled in the art. In this context, for example, full reference can be made to Yamashita, N. et at Vimentin as a poor prognostic factor for triple-negative mammary carcinoma. J. Cancer Res, Can. Oncol. (201), doi:10.1007/s00432-013-1376-6.

Mesenchymal markers preferred according to the invention are vimentin, fibronectin and N-cadherin (Ogunbolude, Y. et al. FRK inhibits breast cancer cell migration and invasion by suppressing Epithelial-mesenchymal transition. Oncotarget (2017). doi:10.18632/oncotarget.22958):

Mesenchymal markers and markers for EMT preferred according to the invention are vimentin, fibronectin. N-cadherin, SNAI1, TWIST1. TWIST2, ZEB1 and ZEB2 (Ogunbolude, Y. et al. FRK inhibits breast cancer cell migration and invasion by suppressing epithelial-mesenchymal transition: Oncotarget (2017). doi:10.18632/oncotarget.22958):

In preferred embodiments of the method according to the invention, the characteristic of the cancer cells is determined in step (b) by quantifying (tumor) stem cell markers.

Suitable (tumor) stem cell markers and methods for their respective quantification are known to a person skilled in the art. In this context, for example, full reference can be made to Li, W. et al. Unraveling the roles of CD44/CD24 and ALDH1 as cancer stem cell markers in tumorigenesis and metastasis. Sci. Rep. (2017). doi:10.1038/s41598-017-14364-2.

(Tumor) stem cell markers preferred according to the invention are CD44/CD24 and ALDH1.

In preferred embodiments of the method according to the invention, the characteristic of the cancer cells is determined in step (b) by quantifying immunomodulating proteins.

Suitable immunomodulating proteins and methods for their respective quantification are known to a person skilled in the art. In this context, full reference can be made, for example, to Bu, X., Yao, Y. & Li, X. Immune checkpoint blockade in breast cancer therapy; in Advances in Experimental Medicine and Biology (2017). doi:10.1007/978-981-10-6020-518.

Immunomodulating proteins preferred according to the invention are check point proteins, e. g. PD-L1, PD-L2; (possibly in co-culture with T cells): PD-1 and CTLA-4,

In preferred embodiments of the method according to the invention, the characteristic of the cancer cells is determined in step (b) by quantifying immune-mediating proteins.

Suitable immune-mediating proteins and methods for their respective quantification are known to a person skilled in the art. In this context, for example, full reference can be made to Vertuani, S. et al. Retinoids Act as Multistep Modulators of the Major Histocompatibility Class I Presentation Pathway and Sensitize Neuroblastomas to Cytotoxic Lymphocytes. Cancer Res. 63:8006-8013 (2003).

An immune-mediating protein preferred according to the invention is the MHC class I protein complex.

In preferred embodiments of the method according to the invention, step (b) comprises incubating a mixture of cancer cells of a cancer cell line and immune cells of an immune cell line with the inhibitor of FKBP1A, the cellular immunogenicity of which is determined as a characteristic of the cancer cells by quantifying the immune cell activation and/or by quantifying the immune cell-mediated cytotoxic effect on cancer cells.

Suitable methods for quantifying the immune cell activation and for quantifying the immune cell-mediated cytotoxic effect on the cancer cells are known to a person skilled in the art.

According to this preferred embodiment, step (c) of the method according to the invention comprises incubating a mixture of cancer cells of the same cancer cell line and immune cells of the same immune cell line as in step (b) in the absence of the inhibitor of FKBP1A and determining the same characteristic of the cancer cells as in step (b) under the same conditions as step (b).

In preferred embodiments of the method according to the invention, the inhibitor provided in step (a) inhibits (i) the expression of FKBP1A and at the same time does not inhibit (ii) the enzymatic activity of FKB1A.

In other preferred embodiments of the method according to the invention, the inhibitor provided in step (a) inhibits (ii) the enzymatic activity of FKB1A and at the same time does not inhibit (i) the expression of FKBP1A.

In preferred embodiments, the method according to the invention comprises the steps of
(a) providing an inhibitor of (i) the expression of FKBP1A;
(b) incubating cancer cells of a cancer cell line, preferably a cancer cell line which is characteristic of triple-negative mammary carcinoma, with the inhibitor of FKBP1A and determining a characteristic of the cancer cells;
(c) incubating cancer cells of the same cancer cell line as in step (b) in the absence of the inhibitor of FKBP1A and determining the same characteristic of the cancer cells as in step (b) under the same conditions as step (b); and
(d) comparing the determined characteristic of the cancer cells according to steps (b) and (C).

According to this preferred embodiment, step (a) comprises the sub-steps
(a₁) providing several test substances;
(a₂) screening the test substances with regard to their inhibitory effect on (i) the expression of FKBP1A; and
(a₃) selecting at least one screened test substance, the inhibitory effect of which is stronger than the inhibitory effect of at least one other screened test substance, and providing this selected test substance as an inhibitor of FKBP1A.

In preferred embodiments of the method according to the invention, the cancer cell line is characteristic of triple-negative mammary carcinoma; the cancer cell line is preferably a cancer cell line of the triple-negative mammary carcinoma.

In preferred embodiments of the method according to the invention, the cancer cell line is a cancer cell line of the triple-negative mammary carcinoma of the mesenchymal stem-like sub-type. The cancer cell line of the triple-negative mammary carcinoma of the mesenchymal stem-like sub-type is preferably selected from MDA-MB-231 and MDA-MB-436; preferably MDA-MB-231.

In preferred embodiments of the method according to the invention, the cancer cell line is a cancer cell line of the triple-negative mammary carcinoma of the basal-like sub-type. The cancer cell line MDA-MB-468 of the triple-negative mammary carcinoma of the sub-type basal-like is preferred.

In preferred embodiments of the method according to the invention, the characteristic (A) determined in step (b) is cellular differentiation status. According to this preferred embodiment of the method according to the invention, the characteristic is preferably determined in step (b) by quantifying mesenchymal markers: preferably by quantifying vimentin.

In further preferred embodiments of the method according to the invention, the characteristic (C) determined in step (b) is cellular immunogenicity. According to this preferred embodiment of the method according to the invention, the characteristic is preferably determined in step (b) by quantifying immunomodulating proteins; preferably by quantifying PD-L1.

In further preferred embodiments of the method according to the invention, the characteristic (E) determined in step (b) is cellular stem cell character. According to this preferred embodiment of the method according to the invention, the characteristic is preferably determined in step (b) by quantifying (tumor) stem cell markers; preferably by quantifying CD44/CD24 and ALDH1.

In further preferred embodiments of the method according to the invention, the characteristic determined in step (b) is the quantifying the expression of FKBP1A. Suitable methods for quantifying the expression of FKBP1A are known to a person skilled in the art, e. g. western blot analysis.

In preferred embodiments, the method according to the invention comprises the steps of
(a) providing an inhibitor of (i) the expression of FKBP1A;
(b) incubating cancer cells of a cancer cell line of triple-negative mammary carcinoma, preferably of the mesenchymal stem-like sub-type, more preferably the cancer cell line MDA-MB-231; with the inhibitor of FKBP1A, and determining a characteristic of the cancer cells, preferably the cellular differentiation status, more preferably by quantifying mesenchymal markers;
(c) incubating cancer cells of the same cancer cell line as in step (b) in the absence of the inhibitor of FKBP1A and determining the same characteristic of the cancer cells as in step (b) under the same conditions as step (b); and
(d) comparing the determined characteristic of the cancer cells according to steps (b) and (c).

In preferred embodiments, the method according to the invention comprises the steps of
(a) providing an inhibitor of (i) the expression of FKBP1A;
(b) incubating cancer cells of a cancer cell line of triple-negative mammary carcinoma, preferably of the mesenchymal stem-like sub-type, more preferably the cancer cell line MDA-MB-231; with the inhibitor of FKBP1A, and determining a characteristic of the cancer cells, preferably cellular immunogenicity, more preferably by quantifying immunomodulating proteins;
(c) incubating cancer cells of the same cancer cell line as in step (b) in the absence of the inhibitor of FKBP1A and determining the same characteristic of the cancer cells as in step (b) under the same conditions as step (b); and
(d) comparing the determined characteristic of the cancer cells according to steps (b) and (c).

In preferred embodiments, the method according to the invention comprises the steps of
(a) providing an inhibitor of (i) the expression of FKBP1A;
(b) incubating cancer cells of a cancer cell line of triple-negative mammary carcinoma, preferably of the mesenchymal stem-like sub-type, more preferably the cancer cell line MDA-MB-231; with the inhibitor of FKBP1A, and determining a characteristic of the cancer cells; preferably cellular stem cell character; more preferably by quantifying (tumor) stem cell markers;
(c) incubating cancer cells of the same cancer cell line as in step (b) in the absence of the inhibitor of FKBP1A and determining the same characteristic of the cancer cells as in step (b) under the same conditions as step (b); and
(d) comparing the determined characteristic of the cancer cells according to steps (b) and (c).

In preferred embodiments, the method according to the invention comprises the steps of
(a) providing an inhibitor of (i) the expression of FKBP1A;
(b) incubating cancer cells of a cancer cell line of triple-negative mammary carcinoma; preferably of the mesenchymal stem-like sub-type, more preferably the cancer cell line MDA-MB-231; with the inhibitor of FKBP1A, and determining a characteristic of the cancer cells, preferably quantifying the expression of FKBP1A;
(c) incubating cancer cells of the same cancer cell line as in step (b) in the absence of the inhibitor of FKBP1A and determining the same characteristic of the cancer cells as in step (b) under the same conditions as step (b); and
(d) comparing the determined characteristic of the cancer cells according to steps (b) and (c).

According to this preferred embodiment; step (a) comprises the sub-steps of
(a₁) providing several test substances;
(a₂) screening the test substances with regard to their inhibitory effect on (i) the expression of FKBP1A,
(a₃) selecting at least one screened test substance, the inhibitory effect of which is stronger than the inhibitory effect of at least one other screened test substance, and providing this selected test substance as an inhibitor of FKBP1A.

Another aspect of the invention relates to the use of an
(A) inhibitor of
   (i) the expression of FKBP1A; and/or
   (ii) the enzymatic activity of FKBP1A; and/or
   (iii) the interaction(s) of FKBP1A with interaction partner(s);

and/or
(B) PROTACs (PROteolysis TArgeting Chimeras), which specifically induces the cellular degradation of FKBP1A, preferably FKBP12 PROTAC RC32 (CAS No.: 2375555-66-9),

for the production of a drug for the treatment of a disease; in particular cancer; preferably triple-negative mammary carcinoma, Her2-positive hormone receptor-negative mammary carcinoma, pancreatic ductal adenocarcinoma (PDAC) or malignant melanoma; particularly preferably from so-called tumor stem cells; particularly preferably the triple-negative mammary carcinoma, preferably the mesenchymal stem-like sub-type.

In a preferred embodiment, the inhibitor inhibits (i) the expression of FKBP1A and at the same time does not inhibit (ii) the enzymatic activity of FKB1A.

In a preferred embodiment, the inhibitor inhibits (ii) the enzymatic activity of FKB1A and at the same time does not inhibit (i) the expression of FKBP1A.

Therapies according to the invention are thus based on different approaches, in particular they intervene in different stages of the formation and effect of FKBP1A in tumor cells. The aim of inhibiting the expression of FKBP1A is that FKBP1A is not formed in the first place or that its concentration is at least reduced. The aim of inhibiting the enzymatic function of FKBP1A is to prevent expressed FKBP1A from developing the enzymatically catalyzed effect. The inhibition of the interaction(s) of FKBP1A with interaction partner(s) aims to suppress secondary processes.

In a preferred embodiment, an inhibitor of the expression of FKBP1A is used in the therapeutic treatment of the disease.

In preferred embodiments of the invention, the inhibitor of the expression of FKBP1A is an FKBP1A-specific siRNA.

Suitable siRNA which is specific for FKBP1A is known to a person skilled in the art and is commercially available, for example siRNA 55205 (ThermoFisher Scientific, Waltham, USA).

In a preferred embodiment, an inhibitor of FKBP1A enzymatic activity is used in the therapeutic treatment of the disease.

In preferred embodiments of the invention, the inhibitor of FKBP1A enzymatic activity is a macrolide, FK506 or a specific non-immunosuppressive inhibitor.

In preferred embodiments of the invention, the inhibitor of FKBP1A enzymatic activity is a specific, non-immunosuppressive, isolated enzyme-targeting inhibitor. The inhibitor is preferably a specific inhibitor of FKBP1A without binding to mTOR or calcineurin and having an immunosuppressive effect.

In a preferred embodiment, an inhibitor of the interaction(s) of FKBP1A with interaction partner(s) is used for the therapeutic treatment of the disease.

Interaction partners of FKBP1A preferred according to the invention are preferably selected with regard to this aspect of the invention from the group consisting of AHSP, cyclophilin-type peptidyl prolyl isomerases, FKBP-type peptidyl prolyl isomerases, inositol triphosphate receptors, MDM2/MDM4, phosphoprotein phosphatases, ryanodine receptors, SF3B4, TGF-beta, TKL Ser/Thr protein kinases and type B carboxylesterases/lipases.

The therapeutic treatment according to the invention preferably takes place as an adjuvant treatment of a cancer. The therapeutic treatment according to the invention is preferably carried out as an adjuvant treatment in an immunotherapy of the cancer and/or to avoid and/or specifically address invading tumor cells, to avoid tumor cell invasion and metastasis formation.

A special form of therapy for the targeted treatment of triple-negative mammary carcinoma is the simultaneous addressing of FKBP1A and the parvulin PIN1 or alternatively the retinoic acid receptors RAR or RXR. For the first time, a strong synergistic effect of inhibition of FKBP1A and inhibition of PIN1 (here using FKBP1A-specific siRNA (or alternatively an FKBP1A-specific inhibitor) and co-incubation with the PIN1-specific inhibitor all-trans retinoic acid, ATRA) was observed in triple-negative mammary carcinoma on, inter alia, the reduction of the tumor stem cell character and the ability to induce apoptosis. On the one hand, such a therapy can be achieved via the combined application of individual specific agents. In this context, on the other hand, a dual-targeting inhibitor can be developed via the concept of pioneering polypharmacology, which as a single agent specifically addresses both peptidyl prolyl cis-trans isomerases. This agent could be composed of, for example, specific inhibitors such as, for example, ElteN378, V-10367 or GPI-1046 against FKBP1A on the one hand and, for example, KPT-6566, ATRA (all-trans retinoic acid) or an ATRA derivative against PIN1, RAR or RXR (Chen, Y. et al. Prolyl isomerase Pin1: a promoter of cancer and a target for therapy, Cell Death and Disease (2018), doi:10.1038/s41419-018-0844-y) on the other, both being either fused or connected via a linker, or in which functional substructures of the inhibitors are “merged” (Ramsay, R. R., Popovic-Nikolic, M. R., Nikolic, K., Uliassi, E. & Bolognesi, M. L. A perspective on multi-target drug discovery and design for complex diseases, Clin. Transl. Med. (2018), doi:10.1186/s40169-017-0181-2). The agents RapaLink-01 (CAS No.: 1887095-82-0) or FKBP12 PROTAC R032 (CAS No.: 2375555-66-9) serve as a chemical example in which an FKBP1A ligand was coupled to a second inhibitor/ligand. A comparable agent can be developed and synthesized by a skilled person with a PIN1 inhibitor such as, for example, KPT-6566 or ATRA (all-trans retinoic acid) or an ATRA derivative. Other linker structures of FKBP1A ligands are known (Kolos, J. M., Voll, A. M., Bauder, M. & Hausch, F. FKBP Ligands—Where We Are and Where to Go? Front. Pharmacol. (2018), doi:10.3389/fphar.2016.01425) and can also be developed by a skilled person with a PIN1 inhibitor such as KPT-6566 or ATRA (all-trans retinoic acid) or an ATRA derivative to form a dual-targeting agent. Exemplary structural formulas for the dual-targeting agents mentioned are shown in FIGS. 7 to 9. The agent according to dependent claim 13 preferably corresponds to one of the agents listed in FIGS. 7 to 9.

FIG. 7 shows an example of the theoretical structure of a dual-targeting agent against FKBP1A and PIM The left box shows the structural component corresponding to rapamycin, the middle box shows an exemplary linker structure and the right box shows an exemplary PIN1 inhibitor. As an alternative to the linker structure given in the middle box, the alternative linker structure can also be used, wherein R₁ represents the rapamycin structure and R₂ represents the PIN1 inhibitor structure. The value for n₁ or n₂ is any positive value including the value 0.

FIG. 8 shows examples, based on FIG. 7, of dual-targeting agents composed of rapamycin and KPT-6556, The chemical bonding of the linker to KPT-6556 or rapamycin is also conceivable to all other possible atoms of the respective partial structures and can be carried out by a skilled person. The value for n₁ or n₂ is any positive value including the value 0.

FIG. 9 shows examples, based on FIG. 7, of dual-targeting agents composed of rapamycin and ATRA. The chemical bonding of the linker to ATRA or rapamycin is also conceivable to all other possible atoms of the respective partial structures and can be carried out by a skilled person. The value for n₁ or n₂ is any positive value including the value 0.

The chemical bonding of a PIN1-specific agent as a dual-targeting agent to a high-affinity ligand of FKBP1A such as rapamycin, as described in [0062] to [0065], and the intracellular bonding of the PIN1-specific agent to FKBP1A effected thereby can also take place for any other agents. Coupling to the cytosolic protein FKBP1A has significant changes to the pharmacokinetics and dynamics of any agent: in tumor cells, especially tumor stem cells, various transporters, inter alia, ABC transporters, are highly overexpressed (Choi, Y. & Yu, A.-M. ABC Transporters in Multidrug Resistance and Pharmacokinetics, and Strategies for Drug Development Curr. Pharm. Des. (2014) doi:10.2174/138161282005140214165212. These transporters ensure rapid removal of the agents from the cell via increased efflux of the agents, so that they can only have a limited and short-term intracellular effect. In addition, there is the metabolism of agents in the cell, which also leads to a loss of effectiveness. A chemical coupling as described in [0062] to [0065] would bind any agent to the cytosolic protein FKBP1A diffusing freely in the cell. However, inter alia due to its size, in contrast to the individual agent, the complex formed is not transported out of the cell, or to a much lesser extent, via the transporters mentioned or is metabolized by cellular processes, which means that a significantly better intracellular effect of the agent can be achieved. The chemical bonding of any agent to a high-affinity ligand of FKBP1A or another cytosolic protein can be introduced in the future as a general novel pharmacological principle with the aim, inter alia, of increasing the effect of any agent in the cell, especially in tumor stem cells or resistant tumor cells.

The therapeutic treatment according to the invention takes place as a treatment of diseases which are preferably selected from cancers and neurodegenerative diseases such as, for example, Alzheimer's disease.

The therapeutic treatment according to the invention preferably takes place as a treatment of cancers which are selected from FKBP1A-positive tumors. The cancer is particularly preferably selected from triple-negative mammary carcinoma, Her2-positive hormone receptor-negative mammary carcinoma and malignant melanoma; in particular triple-negative mammary carcinoma, especially the TNBC mesenchymal stem-like sub-type.

A further aspect of the invention relates to the use of FKBP1A as a prognostic or diagnostic marker for diseases, in particular cancers or neurodegenerative diseases; especially FKBP1A-positive tumors; in particular triple-negative mammary carcinoma, Her2-positive hormone receptor-negative mammary carcinoma or malignant melanoma; in particular triple-negative mammary carcinoma, especially the TNBC mesenchymal stem-like sub-type.

A further aspect of the invention relates to a method for the quantitative detection of FKBP1A in human material for the prognostic and diagnostic assessment of diseases; in particular cancers; preferably cancers with high metastasis potential including for the analysis and diagnosis of tumor invasion and for the prediction of a metastasis tendency; in particular triple-negative mammary carcinoma, Her2-positive hormone receptor-negative mammary carcinoma or malignant melanoma; in particular triple-negative mammary carcinoma, especially the TNBC mesenchymal stem-like sub-type; including the steps of:

(a) providing human material, preferably serum, plasma or tissue; and

(b) quantifying FKBP1A, preferably by determining the concentration.

Step (a) of the method according to the invention for the quantitative detection of FKBP1A in human material preferably includes providing the human material, but not the removal of the material per se. The human material, preferably serum, plasma or tissue, has therefore preferably already been removed beforehand and the human body from which the material was removed does not have to be present in itself for the implementation of the method according to the invention. Thus, step (a) of the method according to the invention includes providing the human material that has already been removed, whereas the removal of the human material per se is preferably not part of the method according to the invention.

Suitable methods for quantifying FKBP1A, in particular for determining the concentration of FKBP1A, are known to a person skilled in the art. In preferred embodiments, step (b) is carried out immunologically by FKBP1A-specific ELISA (serum, plasma) or immunohistologically (tissue).

A further aspect of the invention relates to a method for finding antibodies, proteins or molecules having a specific affinity to FKBP1A, which are suitable for the treatment of diseases, in particular cancers or neurodegenerative diseases, comprising the steps of
(a) providing a library of antibodies, antibody fragments, proteins or protein fragments;
(b) selecting from said library specific FKBP1A-binding antibodies, antibody fragments, proteins or protein fragments;
(c) isolating and sequencing said specific FKBP1A-binding antibodies, antibody fragments, proteins, protein fragments or their encoding genes from step (a); and
(d) expressing the specific FKBP1A-binding antibodies, antibody fragments, proteins or protein fragments in suitable host cells.

Suitable methods for providing antibodies, antibody fragments, proteins or protein fragments, for selecting specific FKBP1A-binding antibodies, antibody fragments, proteins or protein fragments, for isolating and sequencing and for expression are known to a person skilled in the art.

In preferred embodiments; step (b) comprises selecting FKBP1A-binding phages in phage display or selecting by biopanning of libraries of antibodies; antibody fragments; proteins and/or protein fragments.

Another aspect of the invention relates to the use of an antibody, antibody fragment, protein, protein fragment or molecule having specific affinity to FKBP1A for the production of a drug for the treatment of diseases: in particular cancers; in particular FKBP1A-positive tumors; in particular triple-negative mammary carcinoma, Her2-positive hormone receptor-negative mammary carcinoma or malignant melanoma; especially triple-negative mammary carcinoma, especially the TNBCmesenchymal stem-like sub-type.

Another aspect of the invention relates to the use of an antibody; antibody fragment, protein, protein fragment or molecule having specific affinity to FKBP1A for the production of a drug comprising a conjugate of the antibody, protein or molecule with an agent or a special vesicle containing an agent for targeted transport of the agent.

A further aspect of the invention relates to such a conjugate made of antibody, protein or molecule having specific affinity to FKBP1A and agent or agent-containing vesicle.

Another aspect of the invention relates to the use of an antibody; antibody fragment, protein, protein fragment or molecule having specific affinity to FKBP1A for the production of a drug for the specific isolation and characterization of circulating tumor cells; especially FKBP1A-positive tumors; in particular triple-negative mammary carcinoma, Her2-positive hormone receptor-negative mammary carcinoma or malignant melanoma; in particular triple-negative mammary carcinoma, especially the TNBC mesenchymal stem-like sub-type; especially for diagnosis by liquid biopsy.

The following examples serve to illustrate the invention, but are not to be interpreted as limiting.

The following commercial cell lines \, ere used in the experiments below:

| | | Her2 | HR |
|---|---|---|---|
| MDA-MB- | Triple-negative mammary carcinoma | negative | negative |
| 231 | mesenchymal stem-like | | |
| MDA-MB- | Triple-negative mammary carcinoma | negative | negative |
| 436 | mesenchymal stem-like | | |
| MDA-MB- | Triple-negative mammary carcinoma | (positive) | negative |
| 453 | androgen receptor-positive | | |
| MDA-MB- | Triple-negative mammary carcinoma | negative | negative |
| 468 | basal-like | | |
| MCF-7 | luminal breast cell line | negative | positive |
| MCF-10A | benign breast cell line, i.e. non- | positive | positive |
| | malignant | | |
| SKBR3 | mammary carcinoma Her2-positive | positive | negative |
| A375 | malignant melanoma cells | | |

### EXAMPLE 1

MDA-MB-231 (mesenchymal stem-like), MDA-MB-436 (mesenchymal stem-like), MDA-MB-468 (basal-like), MCF-7: (luminal, HR-positive) and MCF-10A (benign breast cell line) (see FIG. 1A) and MDA-MB-453, SKBR3 (Her2-positive) and A375 (melanoma) (see FIG. 1B) were cultivated in uniform growth medium (RPMI-1640 medium, 10% FOS), washed with PBS, lysed and analyzed by western blot.

FIG. 1 shows a western blot analysis of the protein expression of FKBP1A. FIG. 1A shows the result of the western blot analysis for MDA-MB-231 and MDA-MB-436: mesenchymal stem-like; MDA-MB-468: basal-like; MCF-7: luminal, HR-positive; MCF-10A: benign breast cell line. FIG. 1A shows that FKBP1A is highly expressed in triple-negative mammary carcinoma cells but, in contrast, is not detectable in hormone receptor-positive mammary carcinoma cells or in non-malignant cells. FIG. 1A shows that FKBP1A is more highly expressed in “mesenchymal stem-like” triple-negative mammary carcinoma cells (MDA-MB-231 and MDA-MB-436) than in basal-like triple-negative mammary carcinoma cells (MDA-MB-468). FIG. 1B shows the result of western blot analysis for Her2-positive, HR-negative mammary carcinoma cells. FIG. 1B shows the high expression of FKBP1A also in cells of Her2-positive, hormone-receptor-negative mammary carcinoma and malignant melanoma.

### EXAMPLE 2

A culture of MDA-MB-231 cells was incubated with siRNA specific to FKBP1A (35202, Thermo Fisher) for three days, the cells washed with PBS and incubated for another three days without the presence of the siRNA. The cells were then washed again with PBS, lysed, and the expression of FKBP1A was analyzed by western blotting.

FIG. 2 shows the downregulation of FKBP1A protein expression by specific siRNA as a result of western blot analysis of FKBP1A protein expression in MDA-MB-231 cells after incubation with FKBP1A-specific siRNA (S5202, Thermo Fisher).

### EXAMPLE 3

MDA-MB-231 cells were incubated with siRNA specific to FKBP1A (S5202, Thermo Fisher) for three days, the cells washed with PBS and incubated for another three days without the presence of the siRNA. The cells were then washed again with PBS, lysed, the RNA was extracted, cDNA was synthesized, and the expression of different (tumor) stem cell and mesenchymal markers was analyzed by qPCR.

FIG. 3 shows the result of an expression analysis of (tumor) stem cell and mesenchymal markers after FKBP1A knock-down. Expression analysis was performed using quantitative RT-PCR of described tumor stem cell markers and the mesenchymal marker vimentin relative to mock (=1) after FKBP1A knock down using siRNA in MDA-MB-231 cells after 6 days. FIG. 3A shows the result normalized to GAPDH. FIG. 3B shows the result normalized to HRPT1.

### EXAMPLE 4

A culture of MDA-MB-231 cells a incubated with Inhibitor I for six days, changing the medium after three days in the presence of the inhibitor. The cells were then washed with PBS, lysed and the expression of FKBP1A was analyzed by western blotting.

FIG. 4 shows the downregulation of FKBP1A protein expression by inhibitor (I) as a result of a western blot analysis of FKBP1A protein expression in MDA-MB-231 cells after incubation with 10 μM inhibitor (I). FIG. 4 shows that agents can induce a reduction in expression of FKBP1A in triple-negative mammary carcinoma.

### EXAMPLE 5

A culture of MDA-MB-231 cells was incubated with Inhibitor I for six days, changing the medium after three days in the presence of the inhibitor. The cells were then washed again with PBS, lysed, the RNA was extracted, cDNA was synthesized and the expression of different (tumor) stem cell and mesenchymal markers as well as PD-L1 analyzed by qPCR.

FIG. 5 shows the result of an expression analysis of (tumor) stem cell and epithelial differentiation markers after expression reduction of FKBP1A using inhibitor (I). The expression analysis was carried out using quantitative RT-PCR of described (A) tumor stem cell and (B) differentiation markers as well as the (C) immune checkpoint PD-L1 after 6 h incubation of MDA-MB-231 cells with inhibitor (I). FIG. 5 shows that agents in triple-negative mammary carcinoma can bring about a reduction in the expression of (tumor) stem cell markers.

1. A method for finding inhibitors of the peptidyl-prolyl cis-trans isomerase FKBP1A, which are suitable for the treatment of triple-negative mammary carcinoma, comprising steps of:
(a) providing an inhibitor of:
(i) an expression of FKBP1A; and/or
(ii) an enzymatic activity of FKBP1A; and/or
(iii) interaction(s) of FKBP1A with interaction partner(s);
(b) incubating cancer cells of a cancer cell line with the inhibitor of FKBP1A, and determining a characteristic of the cancer cells;
(c) incubating cancer cells of the same cancer cell line as in step (b) in an absence of the inhibitor of FKBP1A and determining the same characteristic of the cancer cells as in step (b) under the same conditions as step (b);
(d) comparing the determined characteristic of the cancer cells according to steps (b) and (c).

2. (canceled)

3. The method according to claim 1, wherein the characteristic determined in step (b) is selected from a group consisting of (A) cellular differentiation status, (B) epithelial characteristics, (C) cellular immunogenicity, (D) apoptosis induction or apoptosis ability, (E) cellular stem cell character, and/or (F) cellular metastatic ability.

4. The method according to claim 1, wherein the inhibitor provided in step (a) inhibits the interaction(s) of FKBP1A with interaction partner(s), with at least one interaction partner being selected from the group consisting of interaction partners which effect a reduction or arrest of (A) the cellular differentiation status, (B) the epithelial characteristics, (C) the cellular immunogenicity (D), the induction of apoptosis or apoptosis ability, (E) the cellular stem cell character, and/or (F) cellular metastatic ability.

5. The method according to claim 1, wherein the inhibitor provided in step (a) inhibits the interaction(s) of FKBP1A with interaction partner(s), at least one interaction partner being selected from the group consisting of interacting proteins, interacting peptides, and interacting nucleic acids.

6. The method according to claim 1, wherein in step (b) the characteristic is determined by quantifying differentiation markers, epithelial markers, (tumor) stem cell markers, immunomodulating proteins, immunomediating proteins or combinations thereof.

7. The method according to claim 1, wherein step (b) comprises incubating a mixture of cancer cells of a cancer cell line and immune cells of an immune cell line with the inhibitor of FKBP1A and wherein, as a characteristic of the cancer cells, their cellular immunogenicity is determined by quantifying the immune cell activation and/or by quantifying the immune cell-mediated cytotoxic effect on the cancer cells; and wherein step (c) comprises incubating a mixture of cancer cells of the same cancer cell line and immune cells of the same immune cell line as in step (b) in the absence of the inhibitor of FKBP1A and determining the same characteristic of the cancer cells as in step (b) under the same conditions as step (b).

8. The method according to claim 1, wherein the inhibitor provided in step (a) inhibits (i) the expression of FKBP1A and at the same time does not inhibit (ii) the enzymatic activity of FKB1A.

9. (canceled)

10. The method according to claim 1, wherein steps (a) is:
providing the inhibitor of (i) the expression of FKBP1A.

11. The method according to claim 10, wherein step (a) comprises the sub-steps of:
(a₁) providing several test substances;
(a₂) screening the test substances with regard to their inhibitory effect on (i) the expression of FKBP1A;
(a₃) selecting at least one screened test substance, the inhibitory effect of which is stronger than the inhibitory effect of at least one other screened test substance, and providing this selected test substance as an inhibitor of FKBP1A.

12. (canceled)

13. The method according to claim 1, wherein the cancer cell line is a cancer cell line of triple-negative mammary carcinoma of the mesenchymal stem-like sub-type.

14. (canceled)

15. The method according to claim 13, wherein the cancer cell line is selected from MDA-MB-231 and MDA-MB-436.

16. (canceled)

17. The method according to claim 3, wherein the characteristic (A) is determined in step (b) by quantifying mesenchymal markers and the characteristic (A) is cellular differentiation status.

18. (canceled)

19. The method according to claim 3, wherein the characteristic (C) is determined in step (b) by quantifying immunomodulating proteins and the characteristic (C) is cellular immunogenicity.

20. (canceled)

21. The method according to claim 3, wherein the characteristic (E) is determined in step (b) by quantifying (tumor) stem cell markers, and the characteristic (E) is cellular stem cell character.

22. The method according to claim 3, wherein the characteristic (F) is determined in step (b) the characteristic is determined by quantifying (tumor) stem cell markers and/or behavior, and the characteristic (F) is cellular metastasis and invasion.

23. The method according to claim 1, wherein the characteristic determined in step (b) is the quantifying the expression of FKBP1A.

24. The method according to claim 1, comprising the steps of:
(a) providing the inhibitor of (i) the expression of FKBP1A;
(b) incubating cancer cells of a cancer cell line of triple-negative mammary carcinoma, of the mesenchymal stem-like sub-type; with the inhibitor of FKBP1A, and determining a characteristic of the cancer cells, wherein the characteristic is the cellular differentiation status, by quantifying mesenchymal markers;
(c) incubating cancer cells of the same cancer cell line as in step (b) in the absence of the inhibitor of FKBP1A and determining the same characteristic of the cancer cells as in step (b) under the same conditions as step (b);
(d) comparing the determined characteristic of the cancer cells according to steps (b) and (c).

25. The method according to claim 1, comprising the steps of:
(a) providing the inhibitor of (i) the expression of FKBP1A;
(b) incubating cancer cells of a cancer cell line of triple-negative mammary carcinoma, of the mesenchymal stem-like sub-type with the inhibitor of FKBP1A, and determining a characteristic of the cancer cells, wherein the characteristic is cellular immunogenicity, by quantifying immunomodulating proteins;
(c) incubating cancer cells of the same cancer cell line as in step (b) in the absence of the inhibitor of FKBP1A and determining the same characteristic of the cancer cells as in step (b) under the same conditions as step (b);
(d) comparing the determined characteristic of the cancer cells according to steps (b) and (c).

26. The method according to claim 1, comprising the steps of:
(a) providing an inhibitor of (i) the expression of FKBP1A;
(b) incubating cancer cells of a cancer cell line of triple-negative mammary carcinoma, of the mesenchymal stem-like sub-type with the inhibitor of FKBP1A, and determining a characteristic of the cancer cells, wherein the characteristic is cellular stem cell character, by quantifying (tumor) stem cell markers;
(c) incubating cancer cells of the same cancer cell line as in step (b) in the absence of the inhibitor of FKBP1A and determining the same characteristic of the cancer cells as in step (b) under the same conditions as step (b);
(d) comparing the determined characteristic of the cancer cells according to steps (b) and (c).

27. The method according to claim 1, comprising the steps of:
(a) providing an inhibitor of (i) the expression of FKBP1A;
(b) incubating cancer cells of a cancer cell line of triple-negative mammary carcinoma, of the mesenchymal stem-like sub-type with the inhibitor of FKBP1A, and quantifying the expression of FKBP1A;
(c) incubating cancer cells of the same cancer cell line as in step (b) in the absence of the inhibitor of FKBP1A and determining the same characteristic of the cancer cells as in step (b) under the same conditions as step (b);
(d) comparing the determined characteristic of the cancer cells according to steps (b) and (c).

28-49. (canceled)
